(19) United States
(12) Patent Application Publication
(10) Pub. No.: US 2025/0006379 A1
(43) Pub. Date: Jan. 2, 2025

(54) METHODS AND SYSTEMS FOR AUTOMATED ANALYSIS OF MEDICAL IMAGES WITH INJECTION OF CLINICAL RANKING

(71) Applicant: ANNALISE-AI PTY LTD, Sydney, New South Wales (AU)

(72) Inventors: Nicolaus CARR, Sydney, New South Wales (AU); Kiet NGUYEN, Sydney, New South Wales (AU); Sam TAM, Sydney, New South Wales (AU)

(21) Appl. No.: 18/705,234

(22) PCT Filed: Oct. 27, 2022
(86) PCT No.: PCT/AU2022/051292

§ 371 Date: Apr. 26, 2024

(30) Foreign Application Priority Data
Oct. 27, 2021 (AU) 2021903432

(51) Int. Cl. G16H 50/30; G16H 30/40

(52) U.S. Cl. CPC G16H 50/30; G16H 30/40

(57) ABSTRACT

A method comprising the steps of: providing a plurality of visual findings in one or more anatomical images of a subject, wherein the plurality of visual findings are generated using a convolutional neural network, CNN, component of a neural network, wherein a 5 subset of the generated visual findings represents a set of priority findings; providing a first classification list for the plurality of visual findings, to associate a first clinical ranking to the set of priority findings, respectively; providing a second classification list wherein the second classification list is user configurable; assigning a clinical ranking to the set of priority findings, using at least one of the first and second classification 10 lists; combining the set of priority findings and their respectively assigned clinical ranking to form triage data; and to generate, using the triage data, an output that represents a re-ordered worklist.

### CROSS-REFERENCE TO RELATED APPLICATIONS

The present application claims priority from Australian Provisional Patent Application No 2021903432 filed on 27 Oct. 2021, the contents of which are incorporated herein by reference in their entirety.

### FIELD OF THE INVENTION

The present invention generally relates to computer-implemented methods for analysing medical images, as well as computing systems, services, and devices implementing the methods. Embodiments of the invention improve on analysis of medical images by providing injection of clinical ranking during automated analysis of medical images employing machine learning techniques, in particular deep learning networks, such as convolutional neural networks, trained using sub-stratification training. Methods, systems, services, and devices embodying the invention find applications, amongst others, in the clinical assessment of chest conditions such as pneumothorax and other radiological findings pertaining to the chest or head.

BACKGROUND TO THE INVENTION

Generally, the manual interpretation of medical images performed by trained experts (such as e.g. radiologists) is a challenging task, due to the large number of possible findings that may be found. For example, the chest x-ray (CXR) is a very commonly performed radiological examination for screening and diagnosis of many cardiac and pulmonary diseases. CXRs are used for acute triage as well as longitudinal surveillance. In other words, a CXR is typically examined for any detectable abnormality in addition to the clinical indication for which it was ordered. This means that radiologists must be alert to identify many different conditions, with a concordant risk that some findings may be missed.

CXRs are particularly difficult to interpret. Additionally, the increasing demand for specialists that are qualified to interpret medical images (i.e. medical imaging specialists or expert radiologists) far outweighs the availability of these specialists. Furthermore, the training of new specialists requires a significant amount of time. As a result, technical operators, such as radiographic technicians/radiographers, are increasingly called upon to provide preliminary interpretations to decrease the waiting time and/or to provide a triage assessment. However, the accuracy and confidence in the work of such technicians is generally inferior to that of highly-trained and highly-experienced specialists.

Empirical training has been used to assess medical imagery, in which mathematical models are generated by learning a dataset. Deep learning is a particularly data-hungry subset of empirical training that is itself a subset of artificial intelligence (AI). Recently, the use of deep learning approaches to generate deep neural networks (DNNs) which are also known as deep learning models, that automate the assessment of CXR images has been suggested. PCT/AU2021/050580 entitled “SYSTEMS AND METHODS FOR AUTOMATED ANALYSIS OF MEDICAL IMAGES” by the same applicant, Annalise-AI Pty Ltd, the contents of which are incorporated in its entirety, describes improved technology in analysing anatomical images using deep learning models.

Prior methods have indicated the presence (and confidence of such prediction) of a radiological finding among a list of findings without any indication of priority of clinical significance of the prediction generated by an AI model 718. There is an ongoing need for improved methods to communicate the results to user systems and user devices in a manner that efficiently produces clinically useful outputs for clinical decision support and exhibiting performance that is non-inferior to conventional unassisted prior methods.

Baltruschat, I. et al., ‘Smart chest X-ray worklist prioritization using artificial intelligence: a clinical workflow simulation’, European Radiology, Vol. 31, No. 6, 2021 discloses a simulation framework that models the current workflow at a university hospital by incorporating hospital-specific CXR generation rates and reporting rates and pathology distribution. In particular, this document discloses ranking of pathologies.

U.S. Pat. No. 9,386,084 B1 discloses systems and methods that allow transfer criteria to be defined based on one or more of several attributes, such as a particular user, site, or device, as well as whether individual images and/or image series are classified as thin slices, and applied to medical images in order to determine which images are downloaded, viewed, stored, and/or any number of other actions that might be performed with respect to particular images.

Annarumma, M. et al, ‘Automated triaging of adult chest radiographs with deep artificial neural networks’, Radiology, Vol. 291, No. 1, 2019 discloses an AI system, based on deep convolutional neural networks (CNNs), for automated real-time triaging of adult chest radiographs on the basis of the urgency of imaging appearances.

In various embodiments the present invention seeks to address, individually and/or in combination, one or more of the foregoing needs and limitations of the prior art.

### SUMMARY OF THE INVENTION

According to a first independent aspect of the invention, there is provided a method comprising the steps of:
providing a plurality of visual findings in one or more anatomical images of a subject, wherein the plurality of visual findings are generated using a convolutional neural network (CNN) component of a neural network, wherein a subset of the generated visual findings represents a set of priority findings;
providing a first classification list for the plurality of visual findings, to associate a first clinical ranking to the set of priority findings, respectively;
providing a second classification list wherein the second classification list is user configurable;
assigning a clinical ranking to the set of priority findings, using at least one of the first and second classification lists;
combining the set of priority findings and their assigned clinical ranking, respectively to form triage data; and
communicating the triage data to a user system configured to process the triage data and to generate, using the triage data, an output that represents a re-ordered worklist.

The set of priority findings represents a subset of the generated visual findings and the triage data (and remainder of the triage process) is only based on this subset of generated visual findings.

In embodiments of the invention, the visual findings may be radiological findings in anatomical images comprising one or more chest x-ray (CXR) images or computed tomography (CT) images.

In a dependent aspect, the second classification list takes precedence over the first classification list. In a further dependent aspect, where the clinical ranking is assigned using both the first and second classification lists, assigning the clinical ranking comprises the steps of: obtaining a first ranking value for a priority finding using the first classification list, obtaining a second ranking value for said priority finding using the second classification list, and providing the second ranking value as the assigned clinical ranking only if the second ranking value is equal to or higher than the first ranking value. The priority findings are then referred to as assignable findings where an assign response is triggered. In other words, the priority findings have been upgraded.

Preferably, the triage data comprises ranking values respectively assigned to each possible “assign response” to ensure the highest ranking value is returned where more than one assignable finding is detected.

The first and second classification lists correspond to two types of clinical rankings, the first one being a ‘default’ priority list that is fixed and non-adjustable by the manufacturer or vendor of the software, whilst the second one is a user defined list that represents a user's worklist. Either or a combination of both may be applied to obtain the clinical ranking. When applied in combination, in preferred embodiments, the second classification list takes precedence, such that a default priority value is only updated if the assigned clinical ranking would result in an upgrade in the user's worklist.

Advantageously, prioritisation of the visual findings is made possible according to embodiments of the invention which generates predictions from one or more deep learning models, looking ahead before the human user (i.e. radiologist), and predicting what is more clinically or medically significant for the human radiologist to consider first temporally. This is in contrast to existing methods which merely assign rankings based on the date/time a patient performs their CXR or other radiological scan, which may be a simple queue based only on time of scan—first come first serve, for example. This is important because some diseases or injuries are time critical and need to be reviewed as soon as possible, otherwise permanent damage to the patient could occur or accelerated deterioration of the patient's medical condition may occur, while other findings are not as time sensitive (i.e. a finding such as tumours). Further advantageously, the second classification is customisable by the user or user system, increasing flexibility and configurability of the system to account for various patient care settings and use cases unique to the user's operational environment.

In a dependent aspect, the triage data comprises an indication of priority findings from the plurality of priority findings that correspond to only one of the group of:

said priority findings, priority findings that have been assigned a clinical value (i.e. assignable findings or priority findings with an assign response), a priority finding that has been assigned a highest clinical ranking. It will be appreciated that the priority finding that has been assigned a highest clinical ranking may be from a plurality of priority findings that have been assigned the same highest clinical ranking (i.e. a group of such priority findings).

In a dependent aspect, the method further comprises the step of: using the triage data to update a user's worklist corresponding to the plurality of visual findings. The user's worklist may comprise at least one study such as a medical study. Preferably the triage data corresponds to a study for a single patient.

In a dependent aspect, updating the user's worklist is configurable to use only one of the group of: said priority findings, priority findings that have been assigned a clinical value (i.e. assignable findings), a priority finding that has been assigned a highest clinical ranking. It will be appreciated that the priority finding that has been assigned a highest clinical ranking may be from a plurality of priority findings that have been assigned the same highest clinical ranking (i.e. a group of such priority findings).

In a dependent aspect, the triage data is provided in a JavaScript Object Notation (JSON) format. In a further dependent aspect, communicating the triage data comprises converting the triage data to a Health Level 7 (HL7) format. This enables the user system to receive the triage data including a field that allows for the assigned clinical ranking to be stored in, enabling automated communication of the findings in order of their priority, in a more robust and reliable manner.

In a dependent aspect the plurality of visual findings and associated first clinical ranking are provided by a server module to an integration layer module. Preferably, the visual findings and associated first clinical ranking are provided using a WebSockets protocol. The integration layer module increases flexibility and configurability of the system 10, whilst the WebSockets protocol provides a security benefit.

In a dependent aspect the integration layer module comprises a database for storing the triage data for a period of time which is configurable by the user. This improves on data security aspects of the system.

In a further aspect, there is provided a system for transmitting triage data for a plurality of visual findings in one or more anatomical images of a subject, wherein the plurality of visual findings are generated using a convolutional neural network (CNN) component of a neural network, the system comprising: at least one processor; and at least one computer readable storage medium, accessible by the processor, comprising instructions that, when executed by the processor, cause the processor to execute a method as described above.

In a further aspect there is provided a non-transitory computer readable storage media comprising instructions that, when executed by at least one processor, cause the processor to execute a method as described above.

Preferred features of each one of the independent claims are provided in the dependent claims.

### BRIEF DESCRIPTION OF THE FIGURES

Aspects of the present invention will now be described, by way of example only, with reference to the accompanying figures, in which:

FIG. 1 is a block diagram of an exemplary architecture of a medical image analysis system embodying the invention;

FIG. 2 is a signal flow diagram illustrating an exemplary method for processing of imaging study results within the embodiment of FIG. 1; and

FIG. 3 is another signal flow diagram illustrating an exemplary method for processing of imaging study results within the embodiment of FIG. 1.

### DETAILED DESCRIPTION

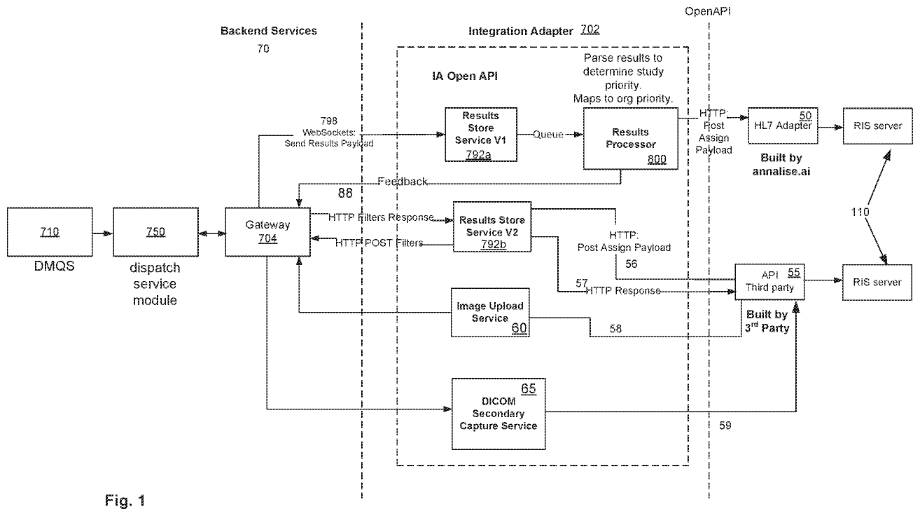

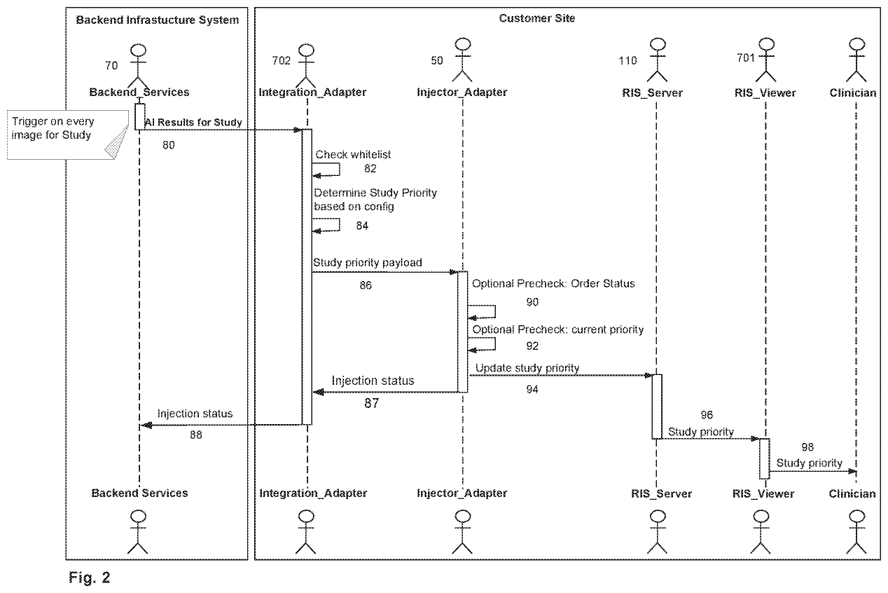

Exemplary systems and methods for analysing radiological findings generated by an AI model on given radiology images (e.g. CXR) are described. Referring to FIGS. 1 and 2, a system 10 comprises modular system components in communication with each other, including a server system 70 configured to send predicted radiological findings associated with a clinical ranking or medical significance rank via an integration layer module 702. The integration layer module 702 includes at least one local database 792a, 792b and a processor 800.

The system 10 further comprises a radiology image analysis server (RIAS) 110. The integration layer 702 may receive worklist priority data from the RIAS 110 representing a user's worklist for a radiologist (i.e. a user), along with associated data which includes a patient identification (ID) and customer account number for example. The predicted radiological findings together with clinical ranking data are provided via the integration layer 702 comprising integrator services, the integration layer 702 connecting to an injection layer 50, the clinical ranking data being processable by the RIAS 110, advantageously communicating to the RIAS 110 the predicted AI radiology findings in a timely manner. This enables a more reliable and safer method of processing predicted radiological findings than known methods, which is particularly important when the radiological findings relate to diseases or injuries which are time critical and need to be identified and confirmed in a timely manner to prevent or minimise permanent injury or accelerated deteriorating medical condition(s) of a patient.

There are two types of clinical rankings that may be applied against a predicted radiological finding generated by the AI model 718: a “priority scale” which represents a first classification or default list (default priority parameter values) and an “assign scale”, which represents a second classification list that is a customer defined list. Either of these types of clinical rankings, or a combination of both may be applied to the radiological findings generated by the AI model 718; when in combination, the “assign scale” takes precedence over the priority scale.

Accordingly, the scales representing worklist priority levels may be customisable per user (i.e. a radiologist or radiographer): for example, user A priority is “Standard, Urgent, Critical” corresponding to ranks 3, 2, 1 respectively which is being processed and mappable by the system 10, while user B may be “Very Low, Med, High, Very High” (ranks 3,2,1, respectively) can also be accommodated for. It will be appreciated that priority levels may be given any suitable values, and can be renamed to any alphanumeric string. Additional levels can also be created.

The code snippets below represent exemplary implementations:

```
″assign″: {
  ″priorities″: [
    {
      ″assignPriorityId″:; 1,
      ″rank″: 1,
      ″priority″: ″10″
    },
    {
      ″assignPriorityId″: 2,
      ″rank″: 2,
      ″priority″: ″20″
    },
    {
      ″assignPriorityId″: 3,
      ″rank″: 3,
      ″priority″, ″30″
    }
  ],
″priority″: ″10″ is the value that is injected into the
RIAS 110 via the injection layer 50.
          10 = Critical
          20 = Urgent
          30 = Standard
″rank″: 1 − internal rank for software to know priority.
″assignPriorityId″: 1 −This id
″labels″: [
    {
      ″label″: ″abdominal_clips″,
      ″groupId″: 2,
      ″displayOrder″: 29,
      ″features″: {
        ″assign″: true,
        ″assist″: true,
        ″assure″: false
      },
      ″assignPriorityId″: 1
    },
    {
      ″label″: ″acute_aortic_syndrome″,
      ″groupId″: 1,
      ″displayOrder″: 43,
      ″features″: {
        ″assign″: true,
        ″assist″: true,
        ″assure″: false
      },
      ″assignPriorityId″: 1
    },
```

Advantageously, the system 10 enables injection of clinical priority levels into the RIAS worklist. This requires the system architecture to be integrated with the RIAS 110 to communicate the predicted radiological findings in a more robust and reliable manner.

Referring to FIG. 2, at step 80, the predicted radiological findings together with assigned clinical ranking data are sent to the integration layer 702 and stored in the database 792a before being queued to the processor 800. The processor 800 may check, at step 82, if the predicted radiological findings are “white-listed” for the RIAS 110. A white-list may be assigned for example using a Digital Imaging and Communications in Medicine (DICOM) tag for the user institution (RIAS) name. It would be possible to select system functionality by enabling or disabling the priority assign functionality; this increases system flexibility and configurability. At step 84, the processor 800 determined the priority of the radiological findings, and maps the findings to the priority, as described above.

At step 56,86, the processor 800 sends the study priority payload comprising the predicted radiological finding and assigned priority data to the injection layer 50 which functions to convert data in a format processable by the RIAS 110. At step 87, the injection status is communicated back to the integration layer 702, and forwarded, at step 88, by the integration layer 702 to the server 70.

If the associated priority for a predicted radiological finding would result in an upgrade in the user's worklist—then a new priority is assigned, at step 84, to the predicted radiological finding. The predicted radiological findings with a new priority assigned are said to be associated with an “assign response”. In this way, no predicted radiological finding will be assigned a downgraded priority in this example. In some embodiments, the priority value is updated at step 84 where the predicted radiological finding is determined to be present in the study. Where more than one predicted radiological finding with an assign response is detected in a study, the highest ranking response, as defined by a worklist user configuration is returned. It will be appreciated that in alternative envisaged processes, a predicted radiological finding may be assigned a downgraded priority; in other words, in alternative envisaged configurations, the first classification list would take precedence over the second classification list representing the default user worklist as defined above.

Accordingly, the integration layer 702 may receive the worklist priority from the RIAS 110 and only apply the new priority value when the original worklist priority is lower. It will be appreciated that, in some cases, the original worklist priority data may not be available; i.e. there is no mechanism to obtain worklist priority levels from the RIAS 110. In these cases, the predicted radiological findings may be selected by the highest available priority, e.g. “Critical”. Accordingly, the priority value injected to the RIAS 110 will only be the highest, thus the original unknown priority level will remain the same or be increased; in other words, there will be no downgrading of study priority. This increases safety and reliability of the system 10.

In preferred embodiments, priority changes are tracked for predicted radiological findings where the original priority value was predicted by the RIAS 110. For example, the number of predicted radiological findings where priority has changed can be tracked, together with the before values predicted by the RIAS 110 and the after (updated values) updated at step 94; the number of predicted radiological findings where priority has not been updated and remains unchanged is also tracked.

Preferably, the data in the assign response indicates the predicted radiological findings that have triggered the assign priority response for one or more of the following scenarios: just the priority findings, just the assignable findings, just the one finding that set the highest priority (or from the highest group if there are more than 1 highest in the group). Preferably, a rank value of each possible assign response should be included to ensure the highest rank response is returned when more than one assign enabled finding is detected.

The study worklist priority update is preferably configurable to respond to: just the priority findings, just the assignable findings, just the one finding that set the highest priority (or from the highest group if there are more than 1 highest in the group), just a subset of studies based on site/facility filtering.

The system 10 further comprises an injection layer 50 to transmit the data to the RIAS 110 in a processable format; in the present example this is a “NextGen™ Connect” module referred to as a MIRTH™ module converting data from a JavaScript Object Notation (JSON) format to Health Level 7 (HL7) format.

Each JSON file received from the integration layer 702 by the injection layer 50 may correspond to a single patient study, e.g. a CXR for one patient. In an example, the JSON payload may contain the following fields: accession number, study instance uid, priority value, rank value, patient information, patient name, patient ID, patient sex, patient date of birth, predicted radiological findings, priority findings, assigned findings, product details, software version, unique device identification (UDI), user guide URL, manufacturer name, manufacturer address.

At steps 90, 92, respectively, the injection layer 50 carries out optional checks of order status and current worklist priority. Examples of order status and worklist priority checks include:
API specific checks.
Active Order Status, such as:
   APPOINTMENT_CONFIRMED
   APPOINTMENT_BOOKED
   ARRIVED
   DISCONTINUED
   SCAN_STARTED
   SCAN_COMPLETED
   IMAGES_VERIFIED
   INTERPRETATION_COMPLETE
   COMPLETE

At step 94, the injection layer 50 communicates the data to predicted radiological findings together with the assigned clinical ranking data to the RIAS 110. At step 96, the RIAS 110 forwards this data to an interactive viewer component 701, which communicates the assigned clinical ranking data to the user at step 98.

The modular components make it highly configurable by users and radiologists in contrast to prior art systems which are rigid and inflexible and cannot be optimised for changes in disease prevalence and care settings. Another benefit of a modular systems architecture comprising asynchronous microservices is that it enables better re-usability, workload handling, and easier debugging processes (the separate modules are easier to test, implement or design). Moreover, the modular aspects allow for users to build an external interface including injection layers/APIs 50, 55. In this way, the system 10 provides an interface specification that allows external applications (patient worklists) to communicate with the system 10 and receive the predicted radiological findings in a more efficient and safe manner, including the order of priority information.

The system 10 also comprises modular components which enable multiple integration and injection pathways to facilitate interoperability and deployment in various existing computing environments such as Radiology Information Systems Picture Archiving and Communication System (RIS-PACS) systems from various vendors and at different integration points such as via APIs 50, 55 or superimposing a virtual user interface element on the display device of the radiology terminals/workstations. The virtual user interface element may be the interactive viewer component 701.

The system 10 provides a plurality of integration pathways via modular subcomponents including: PACS injection, RIS injections, the synchronised viewer component 701, PACS inline frame (iFrame) support, PACS Native AI Support, or a Uniform Resource Locator (URL) hyperlink that re-directs the user to a web viewer on a web page executed in a web browser.

### Integration Layer 702 Configuration

The integration layer 702 comprising integrator services of an integration adapter, comprises one or more software components that may execute at on-premises hardware. The transmission may be processed, controlled and managed by the integration layer 702 for example installed at the radiological clinic or its data centre, or residing at cloud infrastructure. The integration layer 702 may include a library module containing integration connectors, each corresponding to an integration pathway.

Depending on the PACS system that is used by a customer, the library module may receive a request for a particular integration connector for the system 10 of the present invention to interact with the customer via the PACS system. Advantageously, the library module may receive a request for a particular integration connector for the system 10 of the present invention to interact with the customer via the customer's RIS system, for triage injection for re-prioritisation of studies.

Advantageously, the data may be processed within less than 60 seconds from the integration layer 702 receiving the predicted radiological findings for a study, the message being returned to the RIAS 110/PACS.

The integration layer 702 can store patient data in a database 792a, 792b; for example using local meta data cache to store patient data, which may be enabled or disabled. This increases security and safety aspects of the system 10 when it is not desirable to send patient data to the server system 70 or query patient data. Patient data may include patient ID, patient name, data of both, etc. In preferred embodiments, the time data is stored (retention time) is configurable to increase security aspect of the system 10. The integration layer 702 may be configured so that its functionality is time based triggered, wherein a time threshold is a configurable parameter. Alternatively, the RIAS may cause the triggering. The time based triggering allows the integration layer 702 to send a message indicating the study is complete (study complete order) to the receiving server 70 if more than a predetermined number of x seconds have elapsed since the last predicted radiological finding (e.g. a radiological image) has been received for the study. In an example x is 15 seconds; preferably, parameter x is configurable. If another predicted radiological finding (i.e. another radiological image) is received after the study complete message is sent to the receiving server 70, parameter x is reset.

In preferred embodiments, the integration layer 702 can communicate a JSON payload to HTTP/HTTPS servers. This may be a native integration with the RIAS or by use of an injection layer 50, e.g. MIRTH™ module which represents an integration engine located between the integration layer 702 and the RIAS 110 which functions to reorganise the payload, enabling more robust and secure communication with the RIAS 110. The JSON payload with enhanced information can then be processed by the injection layer 50 to communicate the data in a manner processable by the RIAS 110. Preferably the JSON payload includes data that enables extensibility, that is the JSON payload can be customised. Example API specifications are included in Table 2.

Access authentication can be used to allow communication between the processing module 800 and the gateway service 704 of the receiving server 70. These credentials would be requested during the installation through the provisioner tool. Example configurations of the processing module 800 are included in Table 3.

The assign response for each enabled predicted radiological finding is configurable to enable a flexible system for different worklist providers (RIAS 110/PACS). Preferably, the configuration allows, per predicted radiological finding, the worklist fields and values to be set when the finding is present, i.e. a ‘Priority’ field where the response for a detected pneumothorax (one type of chest radiological finding) is set to Critical.

When receiving a DICOM message from the PACS services 65 (step 59), if an assign enabled finding is present in an AI study, the integration layer 702 can update the study worklist priority according to the assign-enabled finding. The study worklist priority is updated when an assign enabled finding is present in the AI study, or if there are more than one assign enabled finding, the highest ranking response (as defined by the customer) is returned.

### Retry Mechanism

The integration layer 702 may comprise a reiteration mechanism referred to as a “retry mechanism” when it is unable to send (at step 58) the image upload request 60 payload to the server 70 or when it is unable to send the JSON payload to injection layer 50,55 (at step 56, 86; optionally a HTTP response is received from the database 792b). This provides for a more robust and reliable system.

For example, the retry mechanism for non-network related errors may be 1+n and consist of:
1. The integration layer 702 shall retry immediately after first time failure.
2. On subsequent failures, the integrator shall retry after x seconds delay for n times. For example, default values may be x=10 seconds and n=3 times. Both x and n values are configurable.
3. If the image upload request has exhausted the retry mechanism, then the payload is put onto the dead letter queue.
4. If the Study Complete has exhausted the retry mechanism, then the payload is put onto the dead letter queue.

For network related errors, the integration layer 702 may retry until successful post can be made for image upload request. For network related errors, the integration layer 702 may retry until successful post can be made for Study Complete upload request. It will be appreciated that the life of messages and queue size of dead letter queue may be configurable to enable flexibility. Examples of network related errors are, internet connectivity issues, server 70 is down and its microservices cannot be accessed (e.g. wrong endpoint URL). Examples of non-network related errors are http 500,502, 503, 504, 418, 420, and 429.

### Injection Layer 50 Configuration

Injection layer 50 is a MIRTH™ module which functions to process the JSON payload, converting data from a JSON format to HL7 format. The HL7 standard includes a field that allows for clinical priority to be stored/assigned. This enables the RIAS 110 to receive the predicted radiological findings and associated clinical ranking in HL7 message format to automatically communicate the studies in their priority order (rank) of e.g. Critical, Urgent, and Standard, enabling the RIAS 110 to process and communicate the predicted radiological findings in a more robust and reliable manner. Examples of envisage configurations for the injection layer 50 are included in Table 1.

### Server Systems and Methods

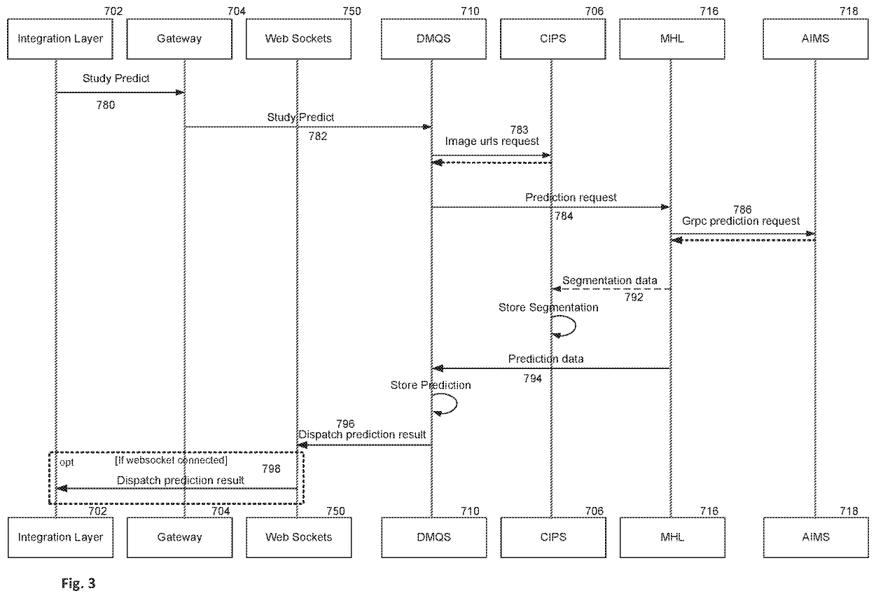

With reference to FIG. 3, a microservice is responsible for acquiring data from the integration layer 702 to send the CXR images to the AI model 718 for generating predicted radiological findings and then sending back the prioritised predicted findings to the integration layer 702. The microservice is also responsible for storing study-related information, CXR images and predicted radiological findings. The microservice provides various secure HTTP endpoints for the integration layer 702 and the viewer component 701 to extract study information to fulfil their respective purposes.

A gateway service 704 provides a stable, versioned, and backward compatible interface to the viewer component 701 and the integration layer 702, e.g. a JSON interface. The gateway 704 provides monitoring and security control, and functions as the entry point for all interactions with a microservice for communicating with an AI model service 718 within the server system 70.

The exemplary method of FIG. 3 is triggered by a study prediction request being sent by the integration layer 702 to the gateway 704. In particular, at step 780 the integration layer 702 sends a “study predict” request comprising an entire study, and which may include associated metadata, i.e. scan, series and CXR images. The request is received by the gateway 704 which, at step 782, forwards the request and other associated data to a distributed message queueing service (DMQS) 710.

At step 783, the request is stored in a database, in this example a cloud imaging processing service (CIPS) 706. The primary functions of the CIPS 706 are to: handle image storage; handle image conversion; handle image manipulation; store image references and metadata to studies and predicted radiological findings; handle image type conversions (e.g. JPEG2000 to JPEG) and store the different image types, store segmentation image results from the AI model(s); manipulate segmentation PNGs by adding a transparent layer over black pixels; and provide open API endpoints for the viewer component 701 to request segmentation maps and radiological images (in a compatible image format expected by the viewer component 701).

The DMQS 710 accepts incoming HTTP requests and listens on queues for message from the gateway 704 and a model handling service (MHS) 716. The DMQS 710 is configured to pass, at step 784, CXR images to the MHS 716 for the model prediction pipeline. The DMQS 710 may store studies, CXR images, and deep learning predictions into a database managed by a database management service (not shown). The DMSQ 710 also manages each study's model findings state and stores the prioritised predicted radiological findings predicted by the AI models, stores errors when they occur in a database, accepts HTTP requests to send study data including model predictions for radiological findings, accepts HTTP requests to send the status of study findings, and forwards CXR images and related metadata to the MHS 716 for processing of the predicted radiological findings.

The MHS 716 is configured to accept DICOM compatible CXR images and metadata from the DMQS 710. The MHS 716 also performs validation, and pre-processing to transform study data into JSON format, which may then be further transformed into a suitable format for efficient communication within the microservice. Then the MHS 716 sends, at step 786 the study data to an AI model service (AIMS) 718 for AI processing, which identifies and returns the predicted radiological findings generated by the deep learning models executed by a machine learning prediction service. The MHS 716 then accepts the predicted radiological findings generated by the deep learning models which are returned via the AIMS 718. The MHS 716 segments (at step 792), validates, and transforms the prioritized predicted radiological findings representing including CXR data together with clinical ranking data predicted by the AI model 718 into JSON format and returns these, at step, 794, to the DMQS 710. For example, each JSON file returned corresponds to a single patient study.

The DMQS 710 sends, at step 796, the CXR data together with the clinical ranking data predicted by the AI model 718 to a dispatch service module 750 which functions to send the CXR data and clinical ranking data to the integration layer 702. Preferably, the integration layer 702 sits behind a firewall, and the dispatch service module returns the CXR data results together with the predicted clinical ranking, at step 798, via a WebSockets service, thereby providing a security benefit.

### Systems

With regard to the preceding overview of the system 10, and other processing systems and devices described in this specification, terms such as ‘processor’, ‘computer’, and so forth, unless otherwise required by the context, should be understood as referring to a range of possible implementations of devices, apparatus and systems comprising a combination of hardware and software. This includes single-processor and multi-processor devices and apparatus, including portable devices, desktop computers, and various types of server systems, including cooperating hardware and software platforms that may be co-located or distributed. Physical processors may include general purpose CPUs, digital signal processors, GPUs, and/or other hardware devices suitable for efficient execution of required programs and algorithms.

Computing systems may include conventional personal computer architectures, or other general-purpose hardware platforms. Software may include open-source and/or commercially available operating system software in combination with various application and service programs. Alternatively, computing or processing platforms may comprise custom hardware and/or software architectures. As previously noted, computing and processing systems may comprise cloud computing platforms, enabling physical hardware resources, including processing and storage, to be allocated dynamically in response to service demands.

Terms such as ‘processing unit’, ‘component’, and ‘module’ are used in this specification to refer to any suitable combination of hardware and software configured to perform a particular defined task. Such a processing unit, components, or modules may comprise executable code executing at a single location on a single processing device, or may comprise cooperating executable code modules executing in multiple locations and/or on multiple processing devices. Where exemplary embodiments are described herein with reference to one such architecture (e.g. cooperating service components of the cloud computing architecture described above) it will be appreciated that, where appropriate, equivalent functionality may be implemented in other embodiments using alternative architectures.

The program code embodied in any of the applications/modules described herein is capable of being individually or collectively distributed as a program product in a variety of different forms. In particular, the program code may be distributed using a computer readable storage medium having computer readable program instructions thereon for causing a processor to carry out aspects of the embodiments of the invention.

Computer readable storage media may include volatile and non-volatile, and removable and non-removable, tangible media implemented in any method or technology for storage of information, such as computer-readable instructions, data structures, program modules, or other data. Computer readable storage media may further include random access memory (RAM), read-only memory (ROM), erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. Computer readable program instructions may be downloaded via transitory signals to a computer, another type of programmable data processing apparatus, or another device from a computer readable storage medium or to an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions, acts, and/or operations specified in the flowcharts, sequence diagrams, and/or block diagrams. The computer program instructions may be provided to one or more processors of a general purpose computer, a special purpose computer, or other programmable data processing apparatus to produce a machine, such that the instructions, which execute via the one or more processors, cause a series of computations to be performed to implement the functions, acts, and/or operations specified in the flowcharts, sequence diagrams, and/or block diagrams.

### Interpretation

Software components embodying features of the invention may be developed using any suitable programming language, development environment, or combinations of languages and development environments, as will be familiar to persons skilled in the art of software engineering. For example, suitable software may be developed using the TypeScript programming language, the Rust programming language, the Go programming language, the Python programming language, the SQL query language, and/or other languages suitable for implementation of applications, including web-based applications, comprising statistical modelling, machine learning, data analysis, data storage and retrieval, and other algorithms. Implementation of embodiments of the invention may be facilitated by the used of available libraries and frameworks, such as TensorFlow or PyTorch for the development, training and deployment of machine learning models using the Python programming language.

It will be appreciated by skilled persons that embodiments of the invention involve the preparation of training data, as well as the implementation of software structures and code that are not well-understood, routine, or conventional in the art of anatomical image analysis, and that while pre-existing languages, frameworks, platforms, development environments, and code libraries may assist implementation, they require specific configuration and extensive augmentation (i.e. additional code development) in order to realize various benefits and advantages of the invention and implement the specific structures, processing, computations, and algorithms described herein with reference to the drawings.

The described examples of languages, environments, and code libraries are not intended to be limiting, and it will be appreciated that any convenient languages, libraries, and development systems may be employed, in accordance with system requirements. The descriptions, block diagrams, flowcharts, tables, and so forth, presented in this specification are provided, by way of example, to enable those skilled in the arts of software engineering, statistical modelling, machine learning, and data analysis to understand and appreciate the features, nature, and scope of the invention, and to put one or more embodiments of the invention into effect by implementation of suitable software code using any suitable languages, frameworks, libraries and development systems in accordance with this disclosure without exercise of additional inventive ingenuity.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Those skilled in the art will be able to make modifications and alternatives in view of the disclosure which are contemplated as falling within the scope of the appended claims. Each feature disclosed or illustrated in the present specification may be incorporated in the invention, whether alone or in any appropriate combination with any other feature disclosed or illustrated herein.

It will be appreciated that the order of performance of the steps in any of the embodiments in the present description is not essential, unless required by context or otherwise specified. Therefore, most steps may be performed in any order. In addition, any of the embodiments may include more or fewer steps than those disclosed.

Additionally, it will be appreciated that the term “comprising” and its grammatical variants must be interpreted inclusively, unless the context requires otherwise. That is, “comprising” should be interpreted as meaning “including but not limited to”.

### Tables

TABLE 1

| | | Injection Layer 50 |
|---|---|---|
| User Integration | Priority Mapping Configuration | Configuration |
| 1. Visage RIS | Can provide more than one level of | 1. Pre-check of order |
| 2. No separate AI | priority | status enabled |
| priority | e.g. | 2. Pre-check of priority |
| 3. No ability to | { | status enabled |
| display predicted | ″assignPriorities″: [ | 3. Inject into existing |
| radiological | { | clinical priority status |
| findings | ″assignPriorityId″ : 0, | |
| | ″rank″:1, | |
| | ″priority″: ″10″ // | |
| | Critical | |
| | }, | |
| | { | |
| | ″assignPriorityId″: 1, | |
| | ″rank″: 2, | |
| | ″priority″: ″20″ // Urgent | |
| | }, | |
| | { | |
| | ″assignPriorityId″: 1, | |
| | ″rank″: 3, | |
| | ″priority″: ″30″ / / | |
| | Standard | |
| | } | |
| | ] | |
| | } | |
| 1. Other HL7 RIAS | Can only provide one level of priority. | 1. Pre-check of order |
| 2. No separate AI | That is the highest level. | status disabled |
| priority | e.g. | 2. Pre-check of priority |
| 3. No ability to | { | status disabled |
| display | ″assignPriorities″: [ | 3. Inject into existing |
| predicted | { | clinical priority |
| radiological | ″assignPriorityId″ : 0, | status |
| findings | ″rank″:1, | |
| | ″priority″: ″10″ / / | |
| | Critical | |
| | } | |
| | ] | |
| | } | |
| 1. Other HL7 RIS | Can provide more than one | 1. Inject into AI |
| 2. Separate AI | level of priority | priority status |
| priority | | |
| 3. No ability to | | |
| display predicted | | |
| radiologic findings | | |
| 1. Other HL7 RIS | Can provide more than one | 1. Inject into AI |
| 2. Separate AI | level of priority | priority status |
| priority | | 2. Provide the |
| 3. Has ability to | | information to |
| display predicted | | injection layer 50 to |
| radiologic findings | | display findings. |
| | | e.g. of payload to |
| | | injection layer 50 |
| | | { |
| | | ″highestPriorityFinding″: { |
| | | ″highestPrioritylabel″: |
| | | ″oesophageal_stent″, |
| | | ″highestPrioritylabel |
| | | Name″: ″Oesophageal |
| | | Stent″ |
| | | }, |
| | | ″allAssignFindings″ : [ |
| | | { |
| | | ″groupId″ : 1, |
| | | ″groupName″: ″Priority″ |
| | | , |
| | | ″assignOrder″: 1, |
| | | ″findings″: [ |
| | | { |
| | | ″label″:″oesophageal_ |
| | | stent″, |
| | | ″labelName″: ″Oesophageal |
| | | Stent″, |
| | | ″displayOrder″: 32, |
| | | ″priorityId″: 10 |
| | | }, |
| | | { |
| | | ″label″: ″lung_collapse″, |
| | | ″labelName″: ″Lung |
| | | Collapse″, |
| | | ″displayOrder″: 64, |
| | | ″priorityId″: 10 |
| | | } |
| | | ] |
| | | } |
| | | ] |
| | | } |

TABLE 2

| POST Request to/priority | Response |
|---|---|
| Result { | Initially response |
| ″accessionNumber″: ″71.15282371″, | could indicate: |
| ″studyInstanceUid″: | 200 OK: Worklist |
| ″1.2.6.1.4.1.14301.74.4.14862474.1″, ″priority″: ″10″, | has been updated |
| ″priorities″: [ | with the new |
| { ″priority″: ″10″, ″rank″:1 }, | priority list or found |
| { ″priority″: ″20″, ″rank″:2 }, | possible precheck |
| { ″priority″: ″30″, ″rank″:3 }, | errors: |
| ], | Study deleted/not |
| ″patient″: { | found/completed |
| ″patientId″: ″71.5736122″, | Priority of the study |
| ″patientName″: ″John Doe″, | is already higher |
| ″patientDob″: ″19860916″, | (currently used in |
| ″patientSex″: ″M″ | ris-injector): |
| }, | { |
| productDetails: { | priorityUpdated, |
| ″productName″: ″Annalise CXR″ | //boolean, |
| ″softwareVersion″: ″2.0.0-003″, | mandatory |
| ″UDI″: ″Null″, | worklistPriority, |
| ″manufactureName″: ″Annalise-AI Pty Ltd″, | //string, |
| ″manufacturerAddress″: ″Level 5, 24 York Street, | optional |
| Sydney, NSW, 2000, Australia″ | worklistStatus, |
| } | //string, |
| productName″ = hardcoded asAnnalise CXR | optional |
| softwareVersion = get from the receiving server 70 | error //string, |
| UDI = hardcode as Null | optional |
| manufactureName = | } |
| hardcode as Annalise-AI Pty Ltd | |
| manufacturerAddress = | |
| hardcode as Level 5, 24 York | |
| Street, Sydney, NSW, 2000, Australia | |
| ″highestPriorityFinding″: { | |
| ″finding″: ″Oesophageal Stent″, | |
| ″priority″: ″10″ | |
| }, | |
| ″allAssignFindings″: [ | |
| { | |
| ″group″: ″Priority″, | |
| ″assignOrder″:1, | |
| ″findings″: [ | |
| { ″finding″: ″Oesophageal Stent″, ″priority″: ″10″ }, | |
| { ″finding″: ″Lung Collapse″, ″priority″: ″20″ } | |
| ] 43 | |
| }, | |
| { | |
| ″group″: ″Other″, | |
| ″assignOrder″: 2, | |
| ″findings″: [ | |
| { ″finding″: ″Rib Resection″, ″priority″: ″20″}, | |
| { ″finding″: ″Shoulder Arthritis″, ″priority″: ″30″}, | |
| ] | |
| } | |
| ], | |
| Update Results processor to generate the following | |
| as part of the payload to the injection layer 50: | |
| 1. Highest priority finding. Note: if there are multiple | |
| priorities with the same rank, then use display | |
| priority to determine the highest. | |
| 2. All relevant findings. | |
| 3. Based on the new payload. The injection layer 50 | |
| will also be able to determine all relevant findings | |
| that is in the high priority group. i.e based on | |
| assignOrder. | |

TABLE 3

| New env variable | Type | Default value? |
|---|---|---|
| Add product details | bool | false |
| add findings | bool | false |
| ris_auth_username | string | None |
| ris_auth_password | string | None |

1. A method comprising the steps of:
providing a plurality of visual findings in one or more anatomical images of a subject, wherein the plurality of visual findings are generated using a convolutional neural network, CNN, component of a neural network, wherein a subset of the generated visual findings represents a set of priority findings;
providing a first classification list for the plurality of visual findings, to associate a first clinical ranking to the set of priority findings, respectively;
providing a second classification list wherein the second classification list is user configurable;
assigning a clinical ranking to the set of priority findings, respectively, using at least one of the first and second classification lists;
combining the set of priority findings and their respectively assigned clinical ranking to form triage data; and
communicating the triage data to a user system configured to process the triage data and to generate, using the triage data, an output that represents a re-ordered worklist.

2. A method according to claim 1, wherein the second classification list takes precedence over the first classification list.

3. A method according to claim 1, wherein the clinical ranking is assigned using both the first and second classification lists, assigning the clinical ranking comprises the steps of: obtaining a first ranking value for a priority finding using the first classification list, obtaining a second ranking value for said priority finding using the second classification list, and providing the second ranking value as the assigned clinical ranking only if the second ranking value is equal to or higher than the first ranking value.

4. A method according to claim 1, wherein the triage data comprises an indication of priority findings from the plurality of priority findings that correspond to only one of the group of:
said priority findings, priority findings that have been assigned a clinical value, a priority finding that has been assigned a highest clinical ranking.

5. A method according to claim 1, wherein the method further comprises the step of: using the triage data to update a user's worklist corresponding to the plurality of visual findings.

6. A method according to claim 5, wherein updating the user's worklist is configurable to use only one of the group of: said priority findings, priority findings that have been assigned a clinical value, a priority finding that has been assigned a highest clinical ranking.

7. A method according to claim 1, wherein the triage data is provided in a JavaScript Object Notation, JSON, format.

8. A method according to claim 7, wherein communicating the triage data comprises converting the triage data to a Health Level 7, HL7 format.

9. A method according to claim 1, wherein the plurality of visual findings and associated first clinical ranking are provided by a server module to an integration layer module.

10. A method according to claim 9, wherein the visual findings and associated first clinical ranking are provided using a WebSockets protocol.

11. A method according to claim 1, wherein the integration layer module comprises a database for storing the triage data for a period of time which is configurable by the user.

12. A system for transmitting triage data for a plurality of visual findings in one or more anatomical images of a subject, wherein the plurality of visual findings are generated using a convolutional neural network, CNN, component of a neural network, the system comprising: at least one processor; and at least one computer readable storage medium, accessible by the processor, comprising instructions that, when executed by the processor, cause the processor to execute a method according to claim 1.

13. A non-transitory computer readable storage media comprising instructions that, when executed by at least one processor, cause the processor to execute a method according to claim 1.
